(19) United States
(12) Patent Application Publication
(10) Pub. No.: US 2011/0082193 A1
(43) Pub. Date: Apr. 7, 2011

(54) TAXANE DERIVATIVE CONTAINING PHARMACEUTICAL COMPOSITION WITH IMPROVED THERAPEUTIC EFFICACY

(76) Inventor: VLADIMIR KYSILKA, Brno (CZ)

(21) Appl. No.: 12/967,247

(22) Filed: Dec. 14, 2010

Related U.S. Application Data
(62) Division of application No. 12/214,496, filed on Jun. 18, 2008.

(30) Foreign Application Priority Data
Dec. 20, 2005 (CZ) PV2005-796

(51) Int. Cl. A61K 31/337; A61P 35/00

(52) U.S. Cl. 514/449

(57) ABSTRACT

The invention relates to a pharmaceutical combination comprising a mixture of (a) at least one taxane derivative and (b) at least one ω-3 poly-unsaturated acid or a derivative thereof wherein the molar ratio of (b) to (a) is not higher than 2. The invention further relates to a liquid pharmaceutical composition comprising (a) an effective amount of at least one taxane derivative, (b) an effective amount of at least one ω-3 poly-unsaturated fatty acid or a derivative thereof and (c) at least one pharmaceutically acceptable carrier and a process for the preparation of the same. The composition can be used for the therapy of cancers which are sensitive to taxane derivatives. The invention also relates to a kit comprising the individual components of the above mentioned composition placed in separate containers.

### FIELD OF THE INVENTION

The invention relates to taxane derivatives containing pharmaceutical compositions with substantially improved therapeutic efficacy and the use of these compositions for the therapy of cancers.

### BACKGROUND OF THE INVENTION

Pharmaceutical compositions comprising taxane drivatives, e.g. paclitaxel, docetaxel, ortataxel or protaxel, are widely used for the therapy of malignant tumor diseases, generically called cancers. Taxane derivatives have a broad anticancer activity due to the multiple mechanisms of action. They are frequently used for the therapy of metastatic breast and ovarian cancers, non-smal cell lung cancer, prostate cancer and other solid cancers, too.

Taxane derivatives have low cancer tissue specificity, which is unfortunately common to all currently used toxic cytostatic agents. High toxicity and low cancer tissue specificity lead to systemic toxicity which is a serious drawback in this cancer therapy. There have been many attempts to improve the low cancer tissue specificity of cytostatic agents, e.g. encapsulation of the agents in microparticles, such as liposomes, chemical conjugation of the agents to the large diversity of natural and synthetic polymer carriers using so-called EPR effect, and chemical conjugation of the agents to low-molecular-weight carriers having specific affinity to structures which are associated with cancer tissues. These attempts have not been too successful because chemical derivatization usually leads to a substantially higher price of the drug and moreover, it reduces the activity of the drug. One partially successful attempt to improve cancer tissue specificity is based on the esterification of paclitaxel or docetaxel by cis-4,7,10,13,16,19-docosahexaenoic acid (DHA). This method is disclosed in PCT/US97/08866, PCT/US97/08792, PCT/US00/96160, US 2002/010208 and WO 2005/042539. In particular, position 2′ of the paclitaxel and docetaxel side chain is preferred for said esterification. These covalent new chemical entities (NCEs) have improved cancer tissue specificity and lower systemic toxicity but unfortunately, they are also markedly less active, which leads to the necessity of increasing the therapeutical dose proposed for clinical trials at least five times. As a consequence, the overall cost of the therapy with the use of these NCEs is at least ten times higher than the therapy with the use of common taxane compositions, e.g. Taxol or Taxotere. It ensues from the above mentioned overview that a simple and effective improvement of the low cancer tissue specificity of taxane derivatives and the improvement of therapeutic efficacy of taxane derivatives have not been successfully solved yet.

In document Menendez J. A. et al.: Oncology Reports, vol. 11, No. 6, June 2004, the in vitro synergic effect of one anticancer agent gamma-linolenic acid and the other anticancer agent docetaxel on selected tumor cell lines is disclosed.

These problems have been solved by the present invention.

### DESCRIPTION OF THE INVENTION

The present invention is based upon the following knowledge:

It has been known that poly-unsaturated fatty acids, in particular ω-3 poly-unsaturated fatty acids, are important cell nutrition components which provide maintenance of cell membrane flexibility and permeability. Moreover, it has been also known that the increased blood content of poly-unsaturated fatty acids leads to about 4-8 fold tumor growth increase, and that tumors accumulate 30-85% of these fatty acids in a single pass of blood. This phenomenon can be explained by the fact that tumor cells are metabolically far more active over the normal tissue. Taxane derivatives have been known to have good affinity to compounds with unsaturated carbon-carbon bonds. Eventually, it has also been known that chemical derivatization of paclitaxel or docetaxel usually leads to the decrease or complete loss of their activity.

The first aspect of the invention is a pharmaceutical combination comprising a mixture of (a) at least one taxane derivative and (b) at least one ω-3 poly-unsaturated fatty acid or a derivative thereof wherein the molar ratio of (b) to (a) is not higher than 2.

The term “a mixture” is used in accordance with its conventional meaning, i.e. it denotes physical or mechanical mixtures in which the components of the mixture do not chemically interact with one another. Whereas any chemical reactions between the taxane derivative (a) and one ω-3 poly-unsaturated fatty acid (b) is practically excluded, strong physical interactions, including van der Waals forces and hydrogen bonds are envisaged between both the species, which is essential for targeting the taxane derivative into the tumor tissue.

A further aspect of the invention is a liquid pharmaceutical composition comprising a mixture of (a) an effective amount of at least one taxane derivative, (b) an effective amount of at least one ω-3 poly-unsaturated fatty acid or a derivative thereof and (c) at least one pharmaceutically acceptable carrier.

In the preferred embodiment the taxane derivative of component (a) is selected from paclitaxel, docetaxel, ortataxel or protaxel.

In another preferred embodiment the ω-3 poly-unsaturated fatty acid of component (b) is α-linolenic acid (ALA).

The claimed solution differs from that disclosed in the aforementioned document Menendez J. A. et al. In contrast to the present invention, gamma-linolenic acid (GLA) used according to said document is not an ω-3 poly-unsaturated fatty acid. In spite of similar denominations of GLA and ALA their effects and uses are quite different. Whereas GLA has an anticancer activity of its own, and it potentiates the effect of docetaxel after its addition to a cell line, cell nutritionally useful ALA and related ω-3 poly-unsaturated fatty acids according to the present invention increase taxane derivative concentration in the tumor tissue and thus enhance its effect. Moreover, Menendez J. A. et al. directly add GLA to the cell line in vitro. The arrangement of the described experiments thus clearly does not rely on any targeting effects of GLA as to docetaxel, which is the gist of the present invention. Thus, even though the chemical structure of GLA and ALA is similar, these compounds can by no means be regarded as equivalent. This is however not at all surprising, since in the field of pharmacotherapy, it is generally known that even small structural changes of the used substances can cause profound differences in terms of their mechanism of action, activity, etc.

Without any intention of being bound by the validity of any proposed theory, the applicant assumes that ω-3 poly-unsaturated fatty acids and/or their derivatives can form sufficiently strong physical conjugates with taxane derivatives without changing their chemical nature. Due to the fact that the resulting physical conjugates comprise the preferred cancer cell nutrition components, they substantially increase cancer tissue specificity of taxane compositions, which in turn leads to substantially increased therapeutic efficacy thereof.

The effective amount of the targeting component (b) is favorably a 1-2 molar amount with respect to the content of a taxane derivative of component (a) of the composition. This amount is sufficient for the formation of physical conjugates with a taxane derivative. The amount of the targeting additives exceeding the molar ratio (b):(a)=2 can be undesirable since in the last mentioned case the targeting additives can compete with the taxane conjugate while providing nutrition to cancer cells without any desirable medical benefit.

The best ω-3 poly-unsaturated fatty acid and/or its derivatives of component (b) for the therapeutic improvement according to the invention is cis,cis,cis-9.12.15-octadecatrienoic acid (α-linolenic acid, ALA) and its ester derivatives. Lower but still great therapeutic improvement can be reached by the use of cis-eicosapentaenoic acid (EPA) and cis-docosahexaenoic acid (DHA). Without any intention of being bound by the validity of any proposed theory, the applicant assumes that the best effect of α-linolenic acid and its esters is based on the fact that α-linolenic acid is a general precursor of all biologically important ω-3 poly-unsaturated fatty acids (PUFAs), including EPA and DHA and so, it is the most attractive nutritive compound for the cancer cell tissues.

Pharmaceutically acceptable carriers (component (c)) include non-ionogenic surfactants or co-solvent systems which comprise non-ionogenic surfactants in combination with a suitable polar solvent or a mixture of polar solvents. As specific examples of such carriers polyoxyethylene sorbitan monooleate or 1:1 (by volume) mixture of polyoxyethylated castor oil and ethanol can be mentioned. A pharmaceutically acceptable carrier can be also a mixture of hydrophobic compounds, as a major component (up to 80%), e.g. glycerides and fatty acid esters, in combination with a hydrophilic component, e.g. ethanol. If a composition according to the invention comprising such carriers is diluted to obtain an infusion solution, an “oil in water” microemulsion comprising a taxane derivative is formed. The “self-emulgating” preconcentrates of this type must not comprise a higher content of ω-3 poly-unsaturated fatty acids and/or their derivatives (component b) than 2 mol per mol of taxane (a) since otherwise the targeting effect may be suppressed or impaired.

According to a further aspect of the invention the aforementioned targeting additives can be added to a composition comprising at least one taxane derivative just before the dilution thereof to obtain an infusion solution. The significant improvement of therapeutic efficacy of the composition according to the present invention is reached by the incorporation of the aforementioned targeting additives in the composition comprising taxane derivatives independently of the time of the addition of the targeting additives to the composition. Even a short contact of the aforementioned targeting additives with taxane derivatives in the composition is sufficient for the formation of their physical conjugates with taxane derivatives and for the increase of therapeutic efficacy of the composition. Thus, a further aspect of the invention is a process for the preparation of the aforementioned pharmaceutical composition which comprises mixing components (a), (b), and (c) and optionally adjusting the concentration of the composition by further dilution to form an infusion solution.

The addition of the aforementioned targeting additives to the composition comprising taxane derivatives just before diluting the composition to obtain an infusion solution is preferred since in this way a problem with the stability of the composition can be avoided.

A further aspect of the invention is a kit for the preparation of an infusion solution which comprises (a) an effective amount of at least one taxane derivative, (b) an effective amount of at least one ω-3 poly-unsaturated fatty acid or a derivative thereof and (c) at least one pharmaceutically acceptable carrier wherein components (a), (b), and (c) are distributed between at least two containers of which one comprises component (a) optionally mixed with a part of component (c) to form a concentrate and the other comprises component (b), optionally mixed with component (c) while an optional further container comprises only component (c).

A further aspect of the invention is the use of the pharmaceutical composition according to the invention for therapy of cancers that are sensitive to taxane derivatives, e.g. breast cancer, ovarian cancer, non-small cell lung cancer, prostate cancer and other solid cancers.

Another advantage of the use of the pharmaceutical composition according to the invention is the fact that only a small amount of targeting compounds is necessary for the substantial increase of the anticancer activity of taxane derivatives. For instance, the addition of equimolar amount of α-linolenic acid with respect to paclitaxel content 6 mg/ml in the composition represents the quantity 1,96 mg/ml and this corresponds to about 0,2% change in the total composition. Because of this small but very important change in the composition this invention makes possible to make use of all advantages and therapeutic experience from commonly used compositions comprising paclitaxel or docetaxel but concurrently, with substantially increased anticancer efficacy. Pharmaceutical composition according to the invention can be also used in a combined cancer therapy with other anticancer compounds.

Pharmaceutical compositions according to the invention are simple, cheap, easy to prepare by common known procedures and easy to use for therapeutic purposes in the same way as known taxane compositions.

The invention is further explained by the following examples. The examples are for illustrative purposes only and shall by no means limit the scope of the invention.

### Example 1.

Preparation of paclitaxel composition with a different nutrition additives

Starting materials:

Ethanol:water content<0,1%

Polyoxyethylated castor oil:Cremophor EL-P (BASF)

Paclitaxel:purity 99,7% (determined by high performance liquid chromatography)

Targeting compound:
a) cis,cis,cis-9,12,15-octadecatrienoic acid (α-linolenic acid, LIN)
b) cis,cis,cis- 9,12,15-octadecatrienoic acid methylester (LIN-ME)
c) cis- 4,7,10,13,16,19-docosahexaenoic acid (DHA)

Procedure:

600 mg of paclitaxel (0,703 mmol) was dissolved in 50 ml of ethanol and 52,7 g (50 ml) of Cremophor EL-P was then added to this solution. One equivalent of LIN (195 mg, 0.7 mmol) was mixed with 0,1 ml of ethanol and the resulting solution was added to the paclitaxel solution. The final paclitaxel composition was passed by means of nitrogen overpressure through a sterilising filter with the porosity 0,2 μm. The sterile solution was subsequently filled into sterile glass vials under laminar flow conditions in an amount 5 ml/vial. Each vial comprised 30 mg of paclitaxel and 9.75 mg of α-linolenic acid. The vials were closed under the nitrogen atmosphere with Omniflex rubber stoppers and secured by aluminium seals. The same procedure was used for the preparation of paclitaxel injections with the addition of two equivalents of LIN, one and two equivalents of LIN-ME and 1 equivalent of DHA, respectively.

The vials containing paclitaxel mixed with the above mentioned additives were used for the tests of therapeutic efficacy without delay—see example 3. If necessary, the vials were stored until use at 5° C. to avoid optional stability problems.

### Example 2

Preparation of a kit comprising a vial with docetaxel concentrate and a vial with an infusion solution solvent containing α-linolenic acid

Starting materials:

Ethanol:water content<0,1%

Targeting compound:α-linolenic acid, purity>99%

TAXOTERE 20 mg concentrate and an infusion solution solvent

Note: The vial with TAXOTERE 20 mg concentrate comprises 0,5 ml of the solution of 20 mg of docetaxel (as anhydrate) in Tween 80. The vial with the infusion solution solvent comprises 1,5 ml 13% w/w solution of ethanol in water for injection.

Procedure:

100 mg of α-linolenic acid (0,359 mmol) was dissolved in 100 μl of ethanol. 10 μl of the resulting solution (0,0359 mmol of α-linolenic acid) was injected through the septum to the solvent vial.

The vials with docetaxel and the vials with the ethanolic solvent containing α-linolenic acid were used for testing therapeutic efficacy without delay. If necessary, they were stored until use at 5° C. to avoid any stability problems.

### Example 3

Therapeutic tests of a paclitaxel composition prepared according to example 1.

Tested injections:

Placebo composition, generic TAXOL composition and paclitaxel compositions with different nutrition additives prepared according to example 1.

Application concentration:

3 portions of the composition diluted with 2-3 portions of saline solution

Tested animal:

Inbred mice DBA2, 8 mice per one tested composition

Process application of the composition to animal:

i.v. (tail), bolus max. 12,5 ml/kg, time of application about 3 minutes

Tested tumor line:

Mouse leukemia L 1210

Process application of tumor line to animal:

s.c., 2×10⁷ of tumor cells

Start of testing of the compositions:

Till tumor volume is about 0,2-0,3 cm³

Testing methodology:
tumor volume evaluation in time (tumor growth curve), up to 30 days
evaluation of tumor growth inhibition (TGI) in % with respect to placebo, up to about 21 days (till mice death).
evaluation of average time survival of mice with tested compositions, up to about 40 days

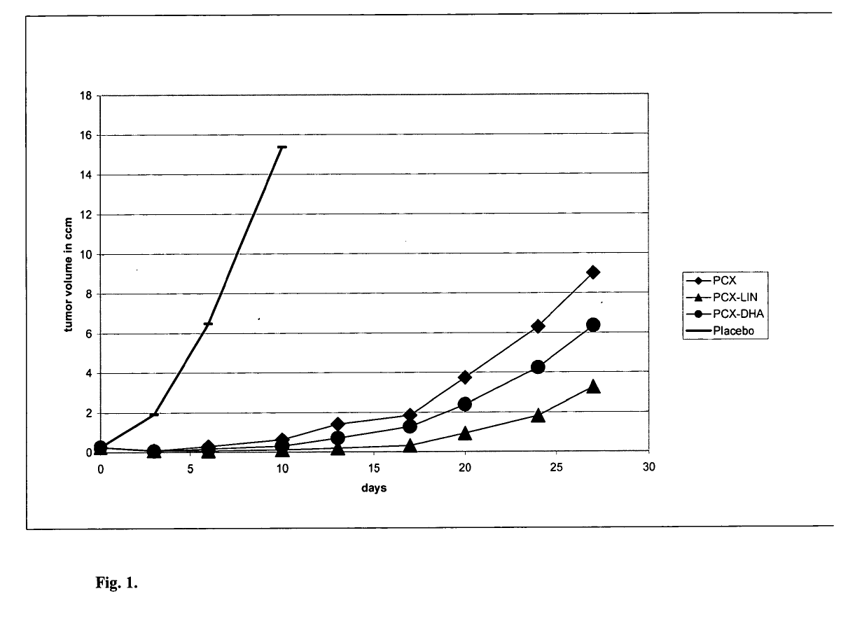

Basic results are summarized in Table 1 and selected results are demonstrated in FIG. 1.

TABLE 1
Dependence of tumor volume of murine leukemia L1210
in cm³ on time in days for paclitaxel compositions
with different targeting compounds according to Example 1

| | | | PCX- | PCX- | PCX- | PCX- | PCX- |
|---|---|---|---|---|---|---|---|
| Days | Placebo | PCX | DHA | LIN | LINME | 2LIN | 2LINME |
| 0 | 0.25 | 0.25 | 0.26 | 0.26 | 0.25 | 0.25 | 0.26 |
| 3 | 1.91 | 0.07 | 0.06 | 0.08 | 0.08 | 0.07 | 0.08 |
| 6 | 6.47 | 0.29 | 0.16 | 0.07 | 0.08 | 0.07 | 0.08 |
| 10 | 15.38 | 0.63 | 0.30 | 0.11 | 0.10 | 0.10 | 0.12 |
| 13 | 26.35 | 1.40 | 0.70 | 0.19 | 0.18 | 0.17 | 0.20 |
| 17 | 29.40 | 1.90 | 1.27 | 0.31 | 0.29 | 0.30 | 0.31 |
| 20 | — | 3.73 | 2.38 | 0.93 | 0.91 | 0.90 | 0.95 |
| 24 | — | 6.29 | 4.23 | 1.82 | 1.75 | 1.81 | 1.90 |
| 27 | — | 9.02 | 6.35 | 3.25 | 3.15 | 3.30 | 3.45 |

Legend to Table 1 and Figure 1:
Placebo = composition without paclitaxel, i.e. Cremophor EL-P with ethanol in 1:1 volume mixture
PCX = the generic paclitaxel composition, i.e. 6 mg paclitaxel/ml of 1:1 volume mixture of ethanol and Cremophor EL-P
PCX-LIN = the generic paclitaxel composition with equimolar additive of cis,cis,cis-9,12,15-octadecatrienoic acid (α-linolenic acid)
PCX-LINME = the generic paclitaxel composition with equimolar additive of cis,cis,cis-9,12,15-octadecatrienoic acid methylester
PCX-DHA = the generic paclitaxel composition with equimolar additive of cis-4,7,10,13,16,19-docosahexaenoic acid
PCX-2LIN = the generic paclitaxel composition with doubled equimolar additive of cis,cis,cis-9,12,15-octadecatrienoic acid (α-linolenic acid)
PCX-2LINME = the generic paclitaxel composition with doubled equimolar additive of cis,cis,cis-9,12,15-octadecatrienoic acid methylester

Average time of mice survival after the application of the composition of paclitaxel with α-linolenic acid was 125% with respect to the application of the generic paclitaxel composition.

### Example 4

Verification of the targeting effect of α-linolenic acid (α-LA) on paclitaxel injections with the use of radioactive ¹³¹I-paclitaxel and a mice model

Two cohorts of mice BALB/c were prepared, each cohort had 12 mice.

Mouse colon cancer, type 4T1, was used. Cancer cells in amount 2×10⁶ were applied s.c. to each mouse. The mice were used for injection tests after tumor volumes about 1 cm³ were reached.

Radioactive ¹³¹I-paclitaxel was prepared by The Institute of Nuclear Research, \check{R}e\check{z} u Prahy.

Radioactive ¹³¹I was chemically bound to the benzene ring in the phenylisoserine side chain of paclitaxel.

Two injection concentrates were freshly prepared before use:

Injection A (5 ml):6 mg ¹³¹I-paclitaxel/1 ml ethanol+CremophorEL mixture 1:1 (v/v)

Injection B (5 ml):6 mg ¹³¹I-paclitaxel+4 mg α-LA/1 ml ethanol+CremophorEL mixture 1:1 (v/v)

Both injection concentrates A and B were diluted with saline in the ratio 1:3 (v/v) to obtain injection boluses A and B. The boluses were homogeneous and clear during 3-5 minutes and they were used for mice tests without delay.

Bolus A was applied in the first cohort with 12 mice. Each mouse received 0.05 ml of bolus A, tail, i.v., during about 1-2 minutes. An average amount of applied activity was 127 kBq/mouse.

Bolus B was applied in the second cohort with 12 mice. Each mouse received 0.05 ml of bolus B, tail, i.v., during about 1-2 minutes. An average amount of applied activity was 130 kBq/mouse.

All mice were sacrificed 5 hour after bolus application. Each tumor was harvested and weighed. The activity of tumor tissue was measured and compared in both cohorts A and B.

The following results were obtained:

An average tumor weight, cohort A: 0.316 g

An average tumor weight, cohort B: 0.322 g

An average proportion of the applied activity deposited in tumor, cohort A: 0.956%

An average proportion of the applied activity deposited in tumor, cohort B: 1.265%

An average proportion of the applied activity per one gram of tumor, cohort A: 3.035%

An average proportion of the applied activity per one gram of tumor, cohort B: 3.930%

Conclusions:

1. The addition of α-linolenic acid to paclitaxel in the molar ratio 2 equivalents α-linolenic acid per 1 equivalent of paclitaxel resulted in the paclitaxel concentration increase by 30% in the tumor tissue. It is a statistically significant increase which demonstrates the targeting effect of α-linolenic acid on a taxane derivative which is translated into substantially increased therapeutic efficacy of a combination according to the invention in which a chemically unchanged taxane derivative is used in the form of a physical mixture with an ω-3 poly-unsaturated acid, such as α-linolenic acid.

2. In general, a relatively small portion of an anticancer drug reached the cancer tissue after 5 hours (about one % of the applied quantity).

1.-17. (canceled)

18. A process for the treatment of malignant tumor diseases comprising administering to a patient suffering from a malignant tumor disease a pharmaceutical composition comprising a mixture of
(a) at least one taxane derivative and
(b) at least one ω-3 poly-unsaturated acid or a derivative thereof wherein the molar ratio of (b) to (a) is not higher than 2, and, optionally, (c) at least one liquid pharmaceutically acceptable carrier.

19. The process according to claim 18 wherein the malignant tumor disease is selected from the group consisting of breast cancer, ovarian cancer, non-small cell lung cancer, prostate cancer and other solid cancers.

20. (canceled)

21. (canceled)

22. The treatment process according to claim 18 wherein the taxane derivative is selected from paclitaxel, docetaxel, ortataxel or protaxel.

23. The treatment according to claim 18, wherein the ω-3 poly-unsaturated acid or a derivative thereof is selected from the group consisting of cis, cis, cis-9, 12, 15-octadecatrienoic acid (α-linolenic acid, ALA) and its ester derivatives, cis-eicosapentaenoic (EPA) and cis-docosahexaenoic acid (DHA).
